(19) United States
(12) Patent Application Publication
(10) Pub. No.: US 2009/0259285 A1
(43) Pub. Date: Oct. 15, 2009

(54) Bifurcated Delivery System

(75) Inventors: Patrick Duane, Ballybrit (IE); Catherine Maresh, Corte Madera, CA (US); Brendan Gallagher, Ballybrit (IE); Terry Guinan, Ballybrit (IE); Colm McCormack, Kilcock (IE); Irene Tully, Ballybrit (IE); Brendan Hanley, Ballybrit (IE)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/100,954

(22) Filed: Apr. 10, 2008

(51) Int. Cl. A61F 2/84; A61F 2/82; A61M 25/09

(52) U.S. Cl. 623/1.11; 623/1.35; 604/164.13

(57) ABSTRACT

A delivery system for a stent to a bifurcated region includes an elongated shaft with first and second branches extending from the elongated shaft. First and second branches have first and second balloons mounted thereon, respectively. A first guidewire extends the entire length of the delivery system and into the first branch for an “over-the-wire” configuration. A second guidewire extends into the second branch, but enters the elongated shaft near a distal end thereof for a “rapid-exchange” configuration. A guideway is formed in the elongated shaft and extends into the first guidewire lumen. The guideway may be forced open by a guide member slidably coupled to the elongated shaft, thereby adjusting the effective over-the-wire length of the first guidewire.

### BACKGROUND OF THE INVENTION

1. Field of the Invention

The present invention relates to catheters used with guidewires in the cardiovascular system and, in particular, to a catheter adapted to deliver a bifurcated prosthesis.

2. Background of the Invention

A wide range of medical treatments have been previously developed using “endolumenal prostheses,” which is intended to mean medical devices which are adapted for temporary or permanent implantation within a body lumen herein. Examples of lumens in which endolumenal prostheses may be implanted include, without limitation: arteries, veins, the gastrointestinal tract, and fallopian tubes. Various types of endolumenal prostheses have also been developed, each providing a uniquely beneficial structure to modify the mechanics of the targeted lumenal wall. For example, various grafts, stents, and combination stent-grafts are well known in the art for implantation within body lumen for providing artificial radial support to the wall tissue which forms the various lumens within the body. More specifically, stents and stent-grafts are often used to provide such support within the blood vessels of the body.

One common type of “stenting” treatment beneficially provides radial support to coronary, peripheral, mesentery or cerebral arteries in order to prevent abrupt reclosure subsequent to recanalization of stenosed vessels, such as by balloon angioplasty or atherectomy (mechanical dilation of stenosed vessel by radial balloon expansion or direct removal of stenotic plaque, respectively). In general, the angioplasty or atherectomy-type recanalization methods reestablish flow to reperfuse tissues downstream of an initial stenosis. Subsequent to such recanalization, however, the dilated lumen of the stenosis site may reocclude, such as by abrupt reclosure (usually due to acute thrombosis or dissected vessel wall flaps transecting the vessel lumen), restenosis (generally considered as a longer term “scarring”-type response to wall injury during recanalization procedures), or spasm (generally considered a response to overdilatation of a vessel and in some aspects may be a form of abrupt reclosure). The implantation of stents to mechanically support the vessel walls at such stenosis sites, either during balloon angioplasty or subsequent to recanalization, is believed to deter the reocclusion of such recanalized vessels which may otherwise occur due to one or more of these phenomena. Various categories of stents have therefore arisen for the primary purpose of providing endolumenal radial support primarily within arteries adjunctively to recanalization.

Stenoses within bifurcation regions of lumens, i.e., points at which a single body lumen branches off or separates into multiple body lumens, more particularly of arterial lumens, have long presented a particular challenge to conventional recanalization techniques, and more particularly to conventional stenting techniques. For example, adjunctively to implanting a stent within a main vessel, which includes a side-branch vessel arising from the main vessel wall along the implanted stent's length, additional stenting of the side-branch vessel may also be required in order to maintain patency of that vessel. The various clinical indications or concerns which are believed to give rise to the desirability of such bifurcation stenting include: mechanical closure of an acutely bifurcating side-branch due to angioplasty of the main vessel or implantation of the main vessel stent; accidentally pushing the carina of the bifurcation point into the main or side-branch vessel during angioplasty; additional stenotic disease in the side-branch vessel; and flow reduction and poor hemodynamics into the sidebranch from the main vessel due to the occlusive presence of the main vessel stents structure in the entrance zone to the side branch. However, it is further believed that conventional stent designs present significant mechanical and procedural challenges to successful stenting of both the main and side-branch vessels at bifurcations of body lumens, and particularly within arterial bifurcations. A thorough discussion of stenting procedures for bifurcated arterial regions may be found in U.S. Pat. No. 6,520,988, the text of which is incorporated herein in its entirety by reference thereto.

One method for delivering stents to a bifurcated region involves the simultaneous delivery of two or more conventional stents or a single, branched stent to the bifurcated region using a single catheter to advance the stent or stents to the desired treatment location. Catheters used for this purpose typically utilize a guide catheter through which two separate balloon catheters are passed, where each balloon catheter is advanced using a separate guidewire. Alternatively, other catheters have a single elongated main body with a branched distal portion, where each branch of the distal portion includes a balloon onto which the stent or stents are loaded for delivery. Each branch of the distal portion is controlled by a separate guidewire, and the elongated body includes one or more lumens through which the multiple guidewires and the inflation hypotube extend.

A common problem in the art is that the multiple guidewires used to advance and manipulate these balloon catheters may entangle with each other and the hypotube while the procedure is being performed. As a result, the complexity and duration of the procedure increase significantly.

U.S. Pat. No. 6,475,208, the text of which is incorporated herein in its entirety by reference thereto, describes catheters used for delivery of stents to bifurcated regions. The catheter described therein employs a bifurcated distal portion and multiple guidewires. In that device, one of the guidewires is used within the catheter in an over-the-wire type arrangement, while the other is used in a rapid-exchange type arrangement, i.e., the guidewire enters the catheter system near the distal portion so that only a small portion of the guidewire is disposed within the catheter.

This configuration makes guidewire exchanges and catheter exchanges difficult. There is a need for a bifurcated delivery catheter system that prevents entanglement between multiple guidewires while allowing for simple guidewire and catheter exchanges.

### SUMMARY OF THE INVENTION

A delivery system for delivering and deploying an endolumenal prosthesis is disclosed. The delivery system includes an elongated shaft, a first guidewire lumen disposed in the elongated shaft, a first guidewire disposed in the first guidewire lumen, and a guideway disposed in the elongated shaft. The guideway is a cut that extends radially into the first guidewire lumen and longitudinally along the elongated shaft. A second guidewire lumen is also disposed in the elongated shaft with a second guidewire disposed therein. A first branch of the elongated shaft extends from the distal end thereof, wherein the first guidewire extends into the first branch. A second branch of the elongated shaft also extends from the distal end thereof, and the second guidewire extends into the second branch. A first balloon is mounted on the first branch and a second balloon is mounted on the second branch, each being fluidly connected to first and second inflation lumens, respectively, disposed in the elongated shaft. A guide member is slidably coupled to the elongated shaft such that the guide member may force open the guideway so as to adjust the over-the-wire length of the first guidewire. The guide member may also include a clamping mechanism that allows a clinician to manipulate an indwelling guidewire.

An embodiment of the delivery system includes an elongated shaft, a first guidewire lumen disposed in the elongated shaft, a first guidewire disposed in the first guidewire lumen, and a first guideway disposed in the elongated shaft, wherein the first guideway extends into the first guidewire lumen. A second guidewire lumen is also disposed in the elongated shaft, with a second guidewire disposed in the second guidewire lumen, and a second guideway disposed in the elongated shaft, wherein the second guideway extends into the second guidewire lumen. A first branch of the elongated shaft extends from the distal end thereof, wherein the first guidewire extends into the first branch. A second branch of the elongated shaft also extends from the distal end thereof, and the second guidewire extends into the second branch. A first balloon is mounted on the first branch and a second balloon is mounted on the second branch, each being fluidly connected to an inflation lumen disposed in the elongated shaft. A guide member is slidably coupled to the elongated shaft to open and close both first and second guideways. The guide member may also include at least one clamping mechanism that allows a clinician to manipulate an indwelling guidewire.

According to an aspect of the present invention, a delivery system for a prosthesis is provided. The delivery system includes an elongated shaft having a proximal end and a distal end, a first guidewire lumen disposed in the elongated shaft, a first guidewire disposed in the first guidewire lumen, and a first guideway disposed in the elongated shaft. The first guideway extends into the first guidewire lumen. The delivery system also includes a second guidewire lumen disposed in the elongated shaft, a second guidewire disposed in the second guidewire lumen, and a first guide member slidably coupled to the elongated shaft. The first guide member is configured to guide the first guidewire through the first guideway and into the first guidewire lumen. A first branch of the elongated shaft extends from the distal end thereof. The first guidewire extends into the first branch. A second branch of the elongated shaft extends from the distal end thereof. The second guidewire extends into the second branch. The delivery system also includes a first balloon disposed on the first branch, a second balloon disposed on the second branch, and an inflation lumen disposed in the elongated shaft. The inflation lumen is fluidly connected to at least the first balloon.

According to an aspect of the invention, there is provided a guidewire placement catheter that includes an elongated shaft having a proximal end and a distal end, a lumen extending from the proximal end to the distal end, the lumen being configured to receive a guidewire, a guideway extending from an outer surface of a proximal section of the elongated shaft to the lumen, and a guide member configured to slide on the outer surface of the proximal portion of the elongated shaft. The guide member has a spreader member constructed and arranged to extend into the guideway and create a gap through which the guidewire may pass into and out of the lumen. The catheter also includes a tracking section connected to the elongated shaft at the distal end of the elongated shaft. The tracking section includes a passageway configured to receive a second guidewire. The passageway is substantially parallel to the lumen.

According to an aspect of the invention, there is provided a method for placing two guidewires into a bifurcated lumen. The method includes advancing a first guidewire into a main branch of a bifurcated lumen, back loading a proximal end of the first guidewire into a tracking section of a guidewire placement catheter, front loading a second guidewire into a lumen of the guidewire placement catheter, advancing the guidewire placement catheter while maintaining the first guidewire position to the bifurcate in the bifurcated lumen, and advancing the second guidwire into a side branch of the bifurcated lumen.

### BRIEF DESCRIPTION OF THE DRAWINGS

Features, aspects and advantages of the present invention will become better understood with reference to the following description, appended claims, and accompanying drawings, which are not to scale.

FIG. 1 illustrates a bifurcated catheter delivery system according to an embodiment of the present invention.

FIG. 1A illustrates the delivery system shown in FIG. 1 with a stent loaded thereon.

FIG. 2 illustrates a transverse cross-sectional view of the delivery system shown in FIG. 1, taken along line A-A.

FIG. 3 illustrates a transverse cross-sectional view of the delivery system shown in FIG. 1, taken along line B-B.

FIGS. 4A-4C illustrate alternative embodiments of a delivery system.

FIGS. 5-7 illustrate various embodiments of transverse cross-sectional views taken along line A-A of alternate embodiments of the delivery system shown in FIGS. 4A-4C.

FIG. 8 is an isometric view of one embodiment of the guide member of FIG. 1.

FIG. 9 is a cross-sectional view of the guide member of FIG. 8 taken on plane C.

FIG. 10 is a cross-sectional view of the guide member of FIG. 8 taken on plane D.

FIG. 11 is an isometric view of an alternative embodiment of the guide member of FIG. 1.

FIG. 12 is an isometric view of an outer tubular member of the guide member of FIG. 11.

FIG. 13 shows an inner body of the guide member of FIG. 11.

FIG. 14 is a cross-sectional view of the inner body of FIG. 13 taken on plane E.

FIG. 15 is an isometric view of another embodiment of the guide member of FIG. 1.

FIG. 16 is a cross-sectional view of the guide member of FIG. 15 taken on plane F.

FIG. 17 is a cross-sectional view of the guide member of FIG. 15 taken on plane G.

FIG. 18 illustrates a bifurcated catheter delivery system according to an embodiment of the present invention being positioned proximal to a bifurcated lumen.

FIG. 19 illustrates the bifurcated catheter delivery system of FIG. 18 with a branch of the system being positioned to enter a side branch of a bifurcated lumen.

FIG. 20 illustrates the bifurcated catheter delivery system of FIG. 19 with the branch being inserted into the side branch of the bifurcated lumen.

FIG. 21 illustrates an embodiment of a guidewire placement catheter.

FIG. 22 illustrates a cross-section of an elongated shaft of the catheter delivery system of FIG. 1 or FIG. 18, or an elongated shaft of the guidewire delivery catheter of FIG. 21 with an embodiment of a shuttle member.

FIG. 23 illustrates the cross-section of the elongated shaft of FIG. 22 with the shuttle member advanced in the elongated shaft.

FIG. 24 illustrates an embodiment of a guidewire placement catheter.

FIG. 25 illustrates the guidewire placement catheter of FIG. 24 being tracked over a guidewire in a main branch of a bifurcated lumen.

FIG. 26 illustrates the guidewire placement catheter of FIG. 25 with a second guidwire being placed in a side branch of the bifurcated lumen.

### DETAILED DESCRIPTION OF THE INVENTION

The present invention is now described with reference to the figures where like reference numbers indicate identical or functionally similar elements. Also in the figures, the left most digit of each reference number corresponds to the figure in which the reference number is first used. While specific configurations and arrangements are discussed, it should be understood that this is done for illustrative purposes only. A person skilled in the relevant art will recognize that other configurations and arrangements can be used without departing from the spirit and scope of the invention.

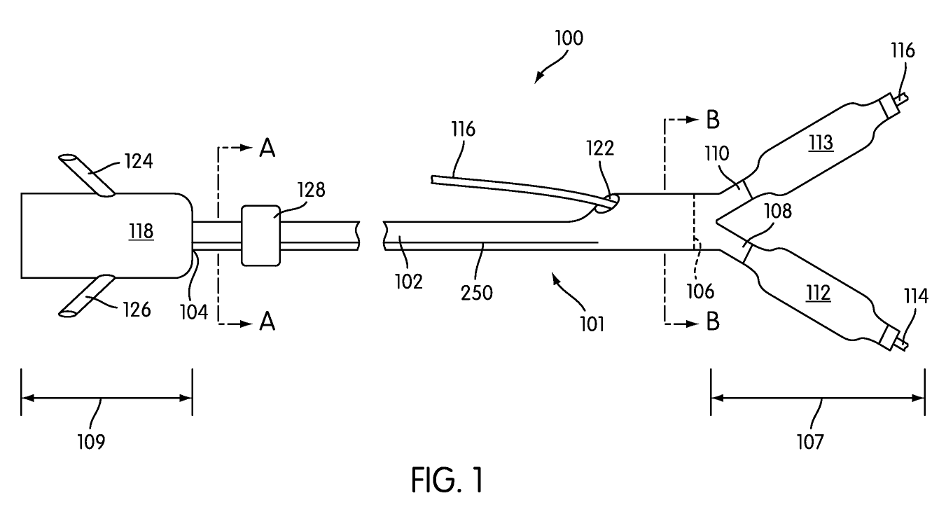

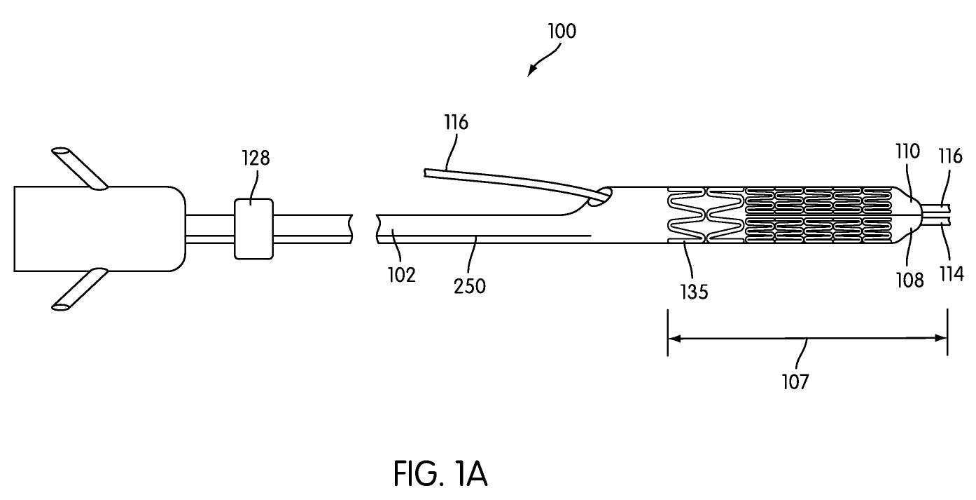

Referring to FIG. 1, a delivery system 100 according to the present invention is shown. Delivery system 100 includes a catheter 101 having an elongated shaft 102 having a proximal end 104 and a distal end 106. In one embodiment, elongated shaft 102 is made of polymeric materials suitable for placement in a patient's body, such as polyvinyl chloride, polyethylene, polyethylene terephthalate, polyamide, or, preferably, polyimide. Further, an optional layer of a stiffer material may be added to or embedded within the main material of elongated shaft 102 to enhance the pushability of delivery system 100. For example, a braid of metal or polymeric filaments could be included. In another embodiment, elongated shaft 102 may be made of other biocompatible materials, for example metals such as stainless steel. Elongated shaft 102 can be manufactured by any method known in the art, such as by extrusion.

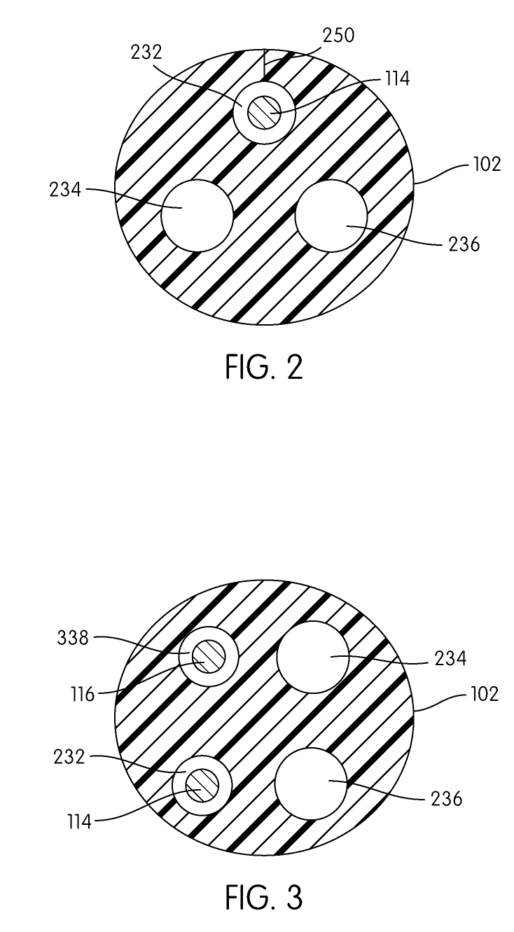

Elongated shaft 102 includes several lumens. As shown in FIGS. 2 and 3, a first guidewire lumen 232, a first inflation lumen 234, and a second inflation lumen 236 extend the entire length of elongated shaft 102. As shown in FIGS. 1 and 3, a second guidewire lumen 338 extends through only a portion of catheter 101, near a distal portion 107.

Distal portion 107 of catheter 101 is configured for the delivery of medical implants, such as stents, to bifurcated vascular regions. Distal portion 107 is formed of a first branch 108 and a second branch 110 leading from distal end 106 of elongated shaft 102. First guidewire lumen 232 extends into first branch 108. A first balloon 112 is mounted around first branch 108. Balloon 112 is similar to other medical balloons known in the art, and may be made of any standard medical balloon material such as nylon, polyethylene terephthalate, polyvinylchloride, PEBAX® polyethylene block amide copolymer, and PELLETHANE® thermoplastic polyurethane elastomer. First balloon 112 may be inflated and deflated through first inflation lumen 234, which extends into first branch 108 to terminate in fluid communication with first balloon 112.

Also, distal portion 107 is shown with first branch 108 and second branch 110 configured in a Y-shaped formation for clarity. In use, first branch 108 and second branch 110 would be held closer together, as shown in FIG. 1A, wherein a stent 135 is shown loaded onto distal portion 107. Stent 135 in this embodiment is designed to be used in bifurcated body lumens, so, similar to distal portion 107, stent 135 expands into a Y-shape. Stent 135 may be one of the type described in U.S. Pat. No. 6,520,988, which is incorporated herein in its entirety by reference thereto. Stent 135 compresses first branch 108 and second branch 110 so that a low profile may be maintained during delivery of stent 135 to the appropriate treatment location.

Second guidewire lumen 338 extends into second branch 110. A second balloon 113 is mounted around second branch 110. Second balloon 113 is made from the same materials as those listed above with respect to first balloon 112. Second balloon 113 may be inflated and deflated through second inflation lumen 236, which extends into second branch 110 to terminate in fluid communication with second balloon 113.

First branch 108 and second branch 110 are formed of any of the materials discussed above with respect to elongated shaft 102. First branch 108, second branch 110 and elongated shaft 102 may be formed from the same or different materials. For example, in one embodiment, first branch 108 and second branch 110 are formed of the same material as elongated shaft 102. In another embodiment, first branch 108 and second branch 110 are formed from the same material, but a different material from that of elongated shaft 102. For example, elongated shaft 102 may be formed from an extruded polymer while first branch 108 and second branch 110 are stainless steel tubes affixed to elongated shaft 102 with an adhesive such as cyanoacrylate adhesive. As a further alternative, each of first branch 108 and second branch 110 may be constructed from a flexible polyethylene sleeve with a flexible polyethylene tube disposed concentrically within the sleeve. In that configuration, the polyethylene tube extends from the respective first or second guidewire lumen 232 and 234 in elongated shaft 102 through distal portion 107.

Distal portion 107 is shown in FIG. 1 with both first branch 108 and first balloon 112 being approximately the same size and configuration as second branch 110 and second balloon 113, respectively, however, such symmetry is not required. In some cases, different sizes and/for configurations may be desired. For example, in many cases, the bifurcated body lumen includes a main vessel and a smaller side-branch vessel. For this situation, first branch 108 and/or first balloon 112 may be significantly smaller in diameter or shorter in length than second branch 110 and/or second balloon 113, or vice versa.

A proximal portion 109 of delivery system 100 includes a hub 118. Hub 118 may be any configuration in the art, such as a luer fitting, and may be made of thermoplastics, polymers, or metals. A first inflation port 124 and a second inflation port 126 are disposed on hub 118. First inflation port 124 is fluidly connected to first inflation lumen 234. Second inflation port 126 is fluidly connected to second inflation lumen 236. First and second inflation ports 124,126 are of a size and shape to be connected to a source of inflation fluid (not shown). The source of inflation fluid may be a syringe, which is inserted into inflation ports 124,126. Other sources of inflation fluid are well-known in the art, such as a hose connected to a fluid reservoir.

A first guidewire 114 extends through first guidewire lumen 232 and into first branch 108. As such, first guidewire 114 extends the entire length of delivery system 100. Such a configuration is known in the art as an “over-the-wire” guidewire configuration. Any material known in the art for use as a guidewire is appropriate for guidewire 114. Examples of such materials include stainless steel, nitinol alloys, or polymeric materials. In one embodiment, guidewire 114 is a solid wire. Alternatively, guidewire 114 may be a hollow tube.

As shown in FIG. 1, in an embodiment of the present invention, second guidewire 116 extends from a guidewire exit port 122 through elongated shaft 102 and into second branch 110. As such, second guidewire 116 is positioned within delivery system 100 only along a relatively short distal length thereof in a “rapid exchange” configuration.

Another feature of delivery system 100 that allows a clinician to maintain control over first guidewire 114 is shown in FIGS. 1 and 2. As shown in FIG. 2, a guideway 250 is formed in elongated shaft 102. In this embodiment, guideway 250 extends from a surface of elongated shaft 102 into first guidewire lumen 232.

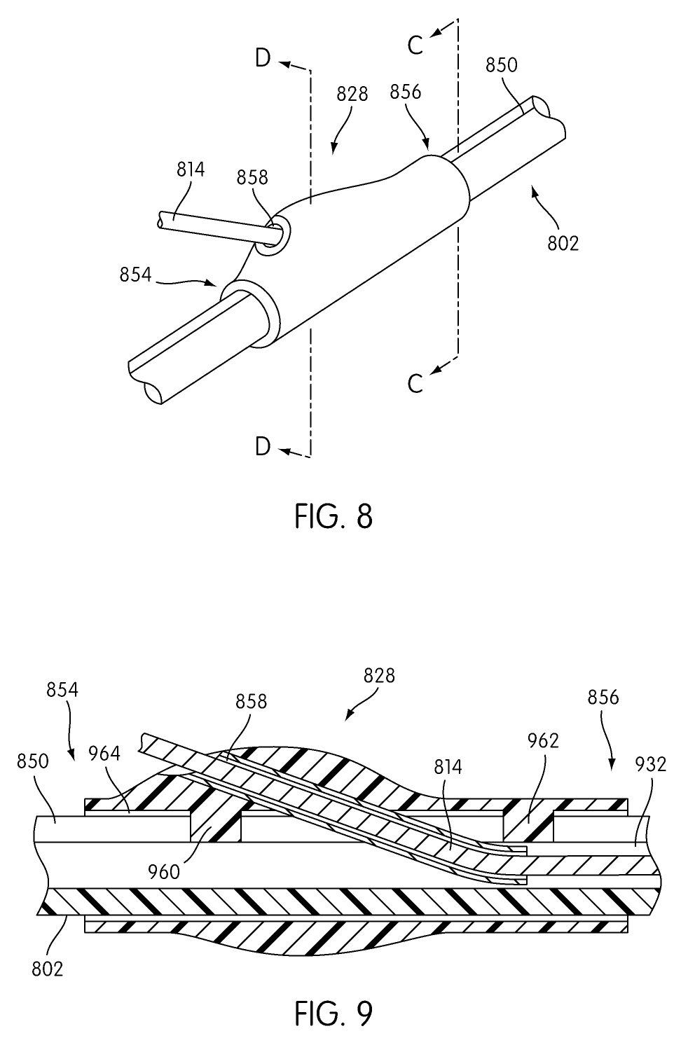

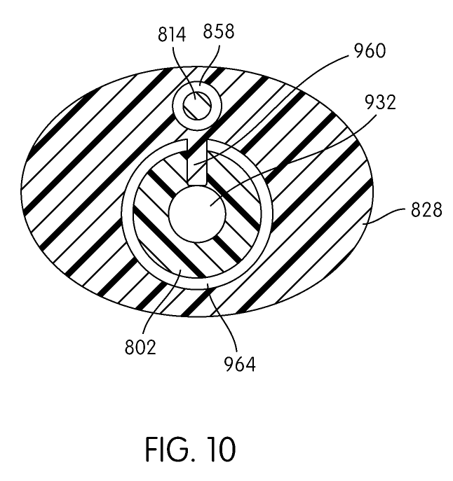

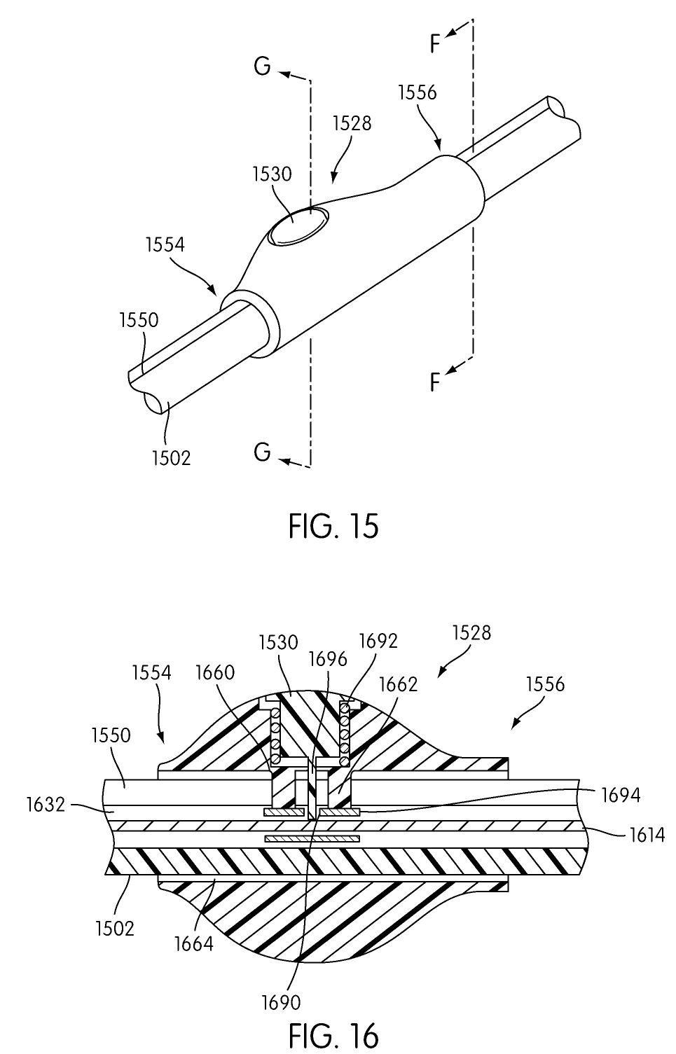

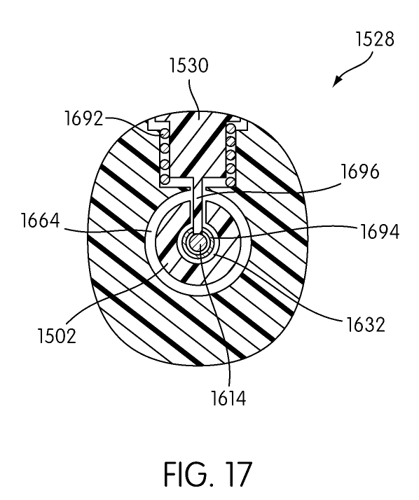

During use, guideway 250 generally remains in a closed position as shown in FIG. 2. However, guideway 250 may be opened by a guide member, shown generically as 128. Guide member 128 is slidably coupled to elongated shaft 102 and allows a clinician to control an indwelling guidewire as it is moved along the length of elongated shaft 102. As discussed below in greater detail, guide member 128 may be used to either adjust the effective over-the-wire length of elongated shaft 102, as described in greater detail below with reference to FIGS. 8-14, or to allow an indwelling guidewire to be moved longitudinally with respect to elongated shaft 102, as described in greater detail below with reference to FIGS. 15-17.

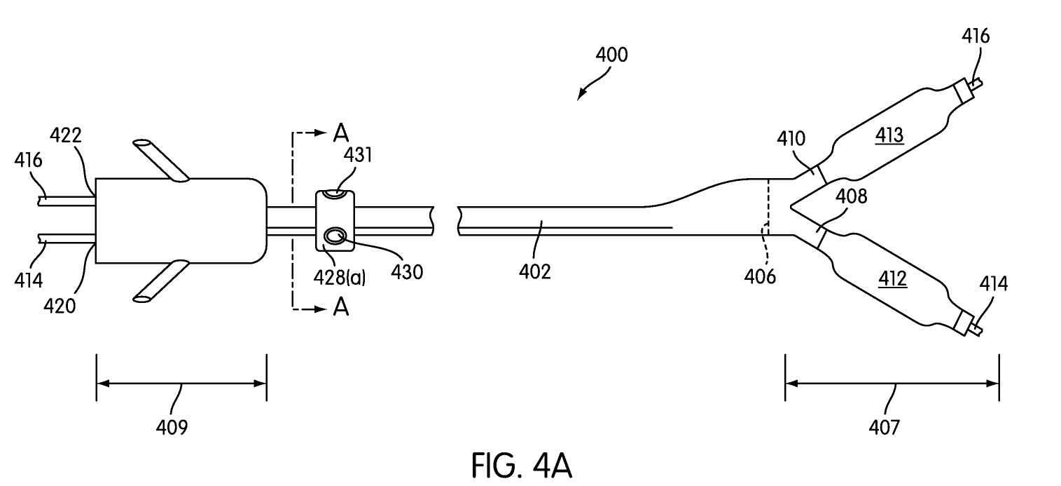

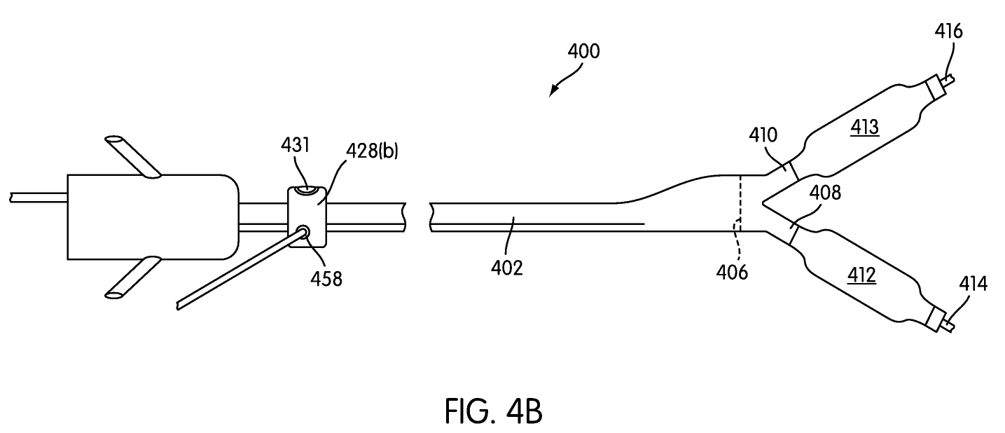

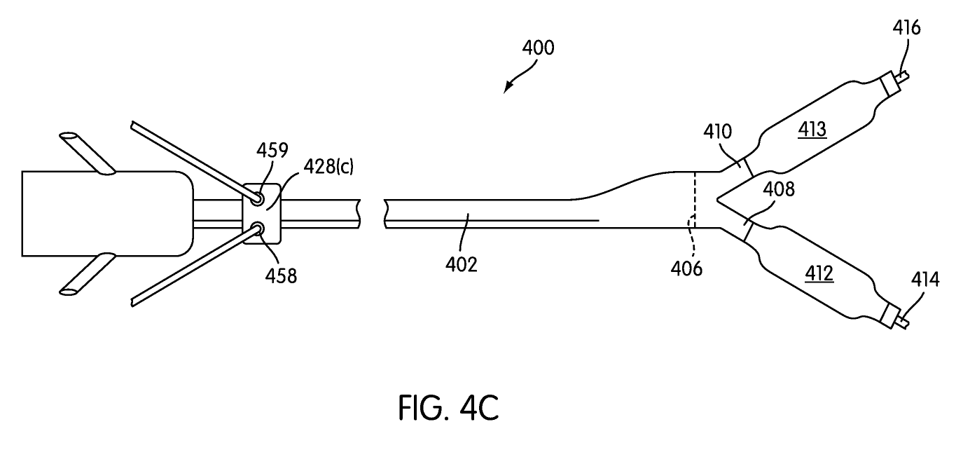

Referring to FIG. 4A, an alternate embodiment of the delivery system is shown. Delivery system 400 includes an elongated shaft 402 similar to elongated shaft 102, as described above. Also, similar to distal portion 107, a distal portion 407 of delivery system 400 includes a first branch 408 having a first balloon 412 mounted thereon and a second branch 410 having a second balloon 413 mounted thereon.

Delivery system 400 includes a first guidewire 414 and a second guidewire 416, which are similar to guidewires 114 and 116 as described above. Both first guidewire 414 and second guidewire 416 may be back loaded into delivery system 400. In an embodiment, first guidewire 414 and second guidewire 416 exit a proximal portion 409 of delivery system 400 through a first guidewire port 420 and a second guidewire port 422. As such, both guidewires 414, 416 extend the entire length of delivery system 400 in an over-the-wire configuration. Alternatively, one or both of the guidewire ports may be provided on guide member 428(a). In that instance, the delivery system would have a variable effective over-the-wire length.

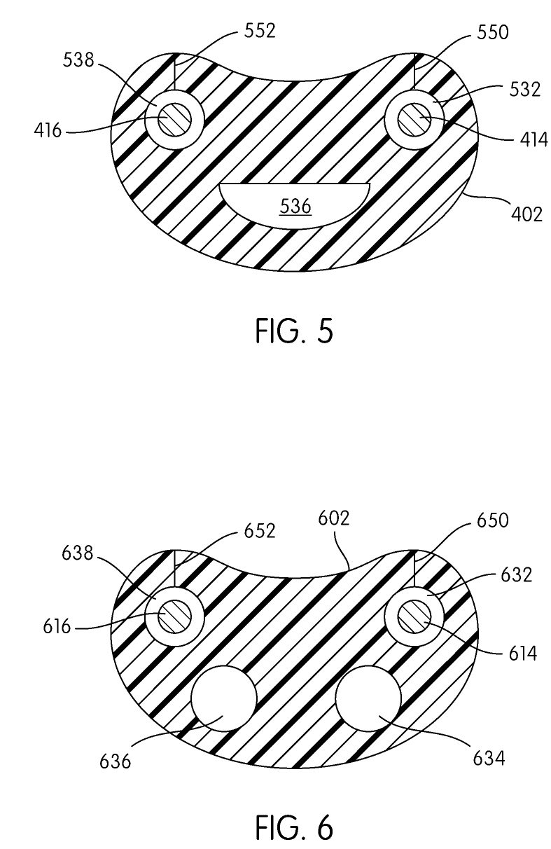

As shown in FIG. 5, first guidewire 414 extends through elongated shaft 402 disposed within a first guidewire lumen 532. Similarly, second guidewire 416 extends through elongated shaft 402 disposed within a second guidewire lumen 538. A first guideway 550 extends from an exterior surface of elongated shaft 402 into first guidewire lumen 532. A second guideway 552 extends from an exterior surface of elongated shaft 402 into second guidewire lumen 538. First and second guideways 550, 552 allow a clinician to control an indwelling guidewire through guide member 428 (shown in FIGS. 4A-4C) in a manner similar to that described above with respect to guide member 128 and as will be described in greater detail below.

Referring to FIG. 4A, a first clamp control member 430 allows a clinician to hold first guidewire 414 in position once guide member 428(a) has been positioned as desired by the clinician. Similarly, a second clamp control member 431 allows a clinician to hold second guidewire 416 in position once guide member 428 has been positioned. First and second clamp control members 430, 431 are similar to clamp control member 1530 (see FIG. 15), described in greater detail below.

As shown in FIGS. 4B and 4C, the guide member included in delivery system 400 is not limited to an embodiment having two clamp control members. For example, FIG. 4B generally shows delivery system 400 with a guide member that includes both clamp control member 431 and a guidewire passageway 458. Such a guide member allows the over-the-wire length of guidewire 414 to be variable while allowing direct positional control over guidewire 116. As a further alternative, guide member 428(c), shown in FIG. 4C, includes guidewire passageway 458 and a second guidewire passageway 459. Guide member 428(c) allows the over-the-wire length of both guidewires 414 and 416 to be variable. In an alternative aspect of the present invention, rather than combining the clamp control members and/or guidewire passageways on one guide member body, independent guide members may be provided where each is dedicated to one guidewire.

As seen in FIG. 5, first and second guidewire lumens 532, 538 and an inflation lumen 536 are disposed within elongated shaft 402. Inflation lumen 536 is used to inflate both first balloon 412 and second balloon 413 (shown in FIGS. 4A-4C), so that both balloons 412, 413 may be inflated simultaneously. In order to conserve space, inflation lumen 536 has a flattened cross-sectional shape, i.e., a semicircular cross-section instead of a circular cross-section. As compared with the stacked configuration of the lumens shown in FIG. 3, guidewire lumens 532, 538, and inflation lumen 536 may be placed closer together in a triangular configuration. As a consequence, the cross-sectional shape of elongated shaft 402 may also be reduced, so that in the embodiment of FIG. 5 the cross-sectional shape of elongated shaft 402 is kidney-shaped instead of the circular shape of elongated shaft 102.

Referring to FIG. 6, another embodiment of the present invention is shown. This embodiment is similar to the embodiment shown in FIGS. 4A-4C, in that a first guidewire 614 and a second guidewire 616 extend the entire length of an elongated shaft 602 through a first guidewire lumen 632 and a second guidewire lumen 638, respectively. In addition, a first guideway 650 and a second guideway 652 similar to the embodiment described above are present in elongated shaft 602. However, as this cross-sectional view of elongated shaft 602 shows, a first inflation lumen 634 and a second inflation lumen 636 are disposed within elongated shaft 602. This allows for individual inflation control of a first balloon which is fluidly connected to first inflation lumen 634 and a second balloon which is fluidly connected to second inflation lumen 636.

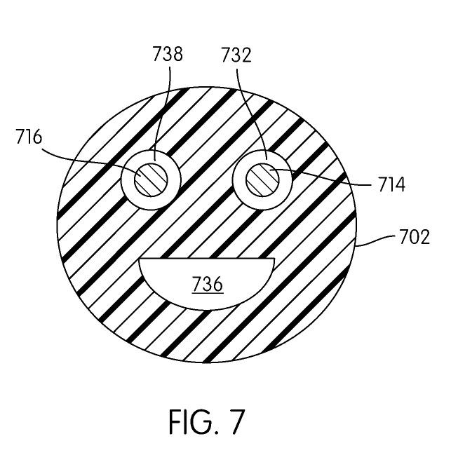

Referring to FIG. 7, another embodiment of the present invention is shown. In the embodiment shown, an elongated shaft 702 has a circular cross-section rather than the kidney shaped cross-sections shown in FIGS. 5 and 6. A first guidewire lumen 732, a second guidewire lumen 738 and an inflation lumen 736 are disposed within elongated shaft 702. As inflation lumen 736 has a semi-circular cross-section, first guidewire lumen 732, second guidewire lumen 738, and inflation lumen 736 may be positioned close together to minimize the outer diameter of elongated shaft 702. In this embodiment, guidewires 714, 716 extend within first and second guidewire lumens 732, 738, which do not include guideways to an exterior surface of elongated shaft 702 and therefore are individually manipulated by more conventional over-the-wire procedures.

As previously mentioned, the guide member may be used to either adjust the effective over-the-wire length of the elongated shaft or to allow an indwelling guidewire to be moved longitudinally with respect to the elongated shaft. A catheter capable of both fast and simple guidewire and catheter exchange that incorporates a guide member that is capable of adjusting the over-the-wire length of the longitudinal shaft is sold by Medtronic Vascular, Inc. of Santa Rosa, Calif. The catheter is sold under the trademarks MULTI-EXCHANGE, ZIPPER MX, ZIPPER, MX and/or MXII (hereinafter referred to as the “MX catheter”) and is disclosed in U.S. Pat. No.: 4,988,356 to Crittenden et al., U.S. Pat. No. 6,800,065 to Duane et al., U.S. Pat. No. 6,893,417 to Gribbons et al., and U.S. Pat. No. 6,905,477 to McDonnell et al.; U.S. Patent Application Publications: 2004-0059369 A1, published Mar. 25, 2004, and 2004-0260329 A1, published Dec. 23, 2004, all of which are incorporated by reference in their entireties.

Although the guide members 128, 428 described above may be used with an MX catheter, FIGS. 8-10 illustrate an embodiment of a guide member 828 that may also be used with an MX catheter. Guide member 828 has proximal and distal ends, 854 and 856 respectively. A catheter receiving bore 964 extends longitudinally through guide member 828 from guide member proximal end 854 to distal end 856. Guide member 828 includes a proximal spreader member 960 and a distal spreader member 962 extending radially into catheter receiving bore 964. The pair of spreader members serve to locally spread open a guideway 850 when guide member 828 is slideably mounted on an elongated shaft 802. A guidewire passageway 858 extends through guide member 828 such that the distal-most end of guidewire passageway 858 intersects with catheter receiving bore 964 at a shallow angle, preferably ranging from 3° to 15°, at a location between proximal spreader member 960 and distal spreader member 962. As distinguished from proximal spreader member 960, distal spreader member 962 should not project into guidewire lumen 932, where it could interfere with guidewire 814.

Guide member 828 may be molded from a rigid plastic material, such as nylon or a nylon based co-polymer, that is preferably lubricious. Alternatively, guide member 828 may be made of a suitable metal, such as stainless steel, or guide member 828 may have both metal components and plastic components. For ease in manufacturing, guide member 828 may be comprised of molded parts that snap-fit together to form the final configuration.

Elongated shaft 802 and guidewire 814 both extend through guide member 828 and merge so that guidewire 814 extends into guidewire lumen 932, as shown in FIG. 9. Elongated shaft 802 extends through catheter receiving bore 964 of guide member 828, engaging proximal spreader member 960 therein. Proximal spreader member 960 extends through guideway 850 in elongated shaft 802 to spread guideway 850 apart. Guidewire 814 may extend through guidewire passageway 858 into catheter receiving bore 964 and further into guidewire lumen 932 through the spread open guideway 850. As elongated shaft 802 is drawn through guide member 828, the once spread open guideway 850 is drawn closed under the influence of the inherent resiliency of elongated shaft 802, thus enclosing guidewire 814 within guidewire lumen 932.

In an alternative maneuver, guidewire 814 may be inserted or removed through guidewire passageway 858, while guide member 828 is held stationary with respect to elongated shaft 802. In this fashion, a guidewire exchange may be performed. In yet another procedure, guidewire 814 and elongated shaft 802 can be held relatively still while guide member 828 is translated, thus “unzipping” and “zipping” guidewire 814 and elongated shaft 802 transversely apart or together, depending on which direction guide member 828 is moved.

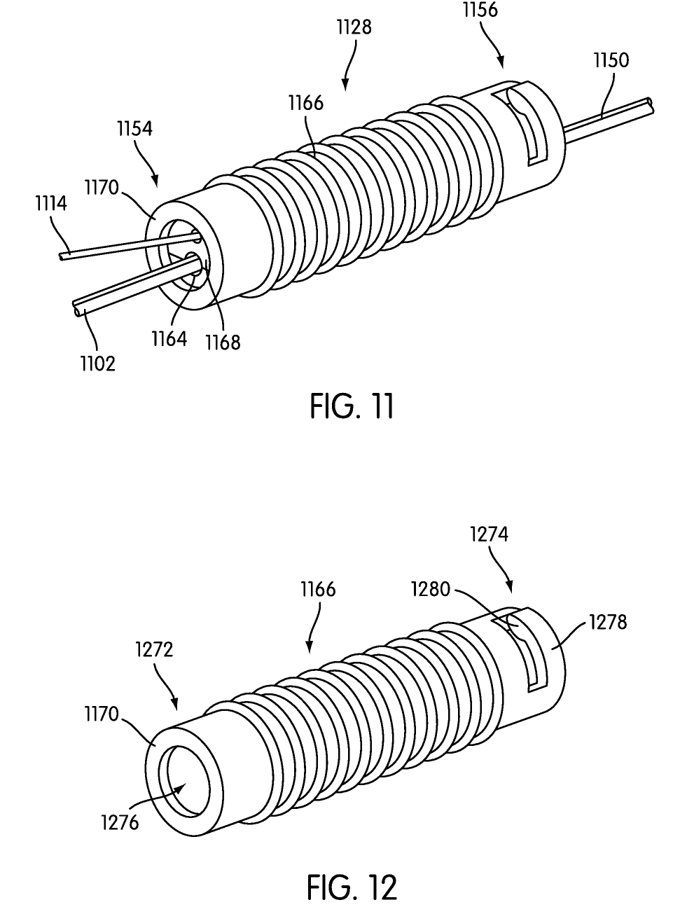

FIGS. 11-14 show an alternative embodiment of a guide member 1128 that may be used with an MX catheter. Guide member 1128 is slidably mounted on an elongated shaft 1102 and has a proximal end 1154 and a distal end 1156. Guide member 1128 has an outer tubular member 1166 with proximal and distal ends, 1272 and 1274 respectively, and a longitudinal bore 1276 sized to receive an inner body 1168. The outer tubular member 1166 freely rotates about inner body 1168 but is coupled to resist relative axial movement between outer tubular member 1166 and inner body 1168, as shown in FIG. 12. A stop shoulder 1170 positioned on proximal end 1272 of the outer tubular member 1166 consists of an annular wall that extends radially into longitudinal bore 1276. The stop shoulder 1170 prevents inner body 1168 from slipping out of outer tubular member 1166 through proximal end 1272 of outer tubular member 1166.

Two retaining arms 1278 are disposed on distal end 1274 of outer tubular member 1166. Retaining arms 1278 consist of two arcuate arms that form a portion of outer tubular member 1166. Each arm 1278 contains a tab 1280 that extends into longitudinal bore 1276 of outer tubular member 1166 at its distal end 1274. When guide member 1128 is assembled, tabs 1280 prevent inner body 1168 from slipping out of outer tubular member 1166 through its distal end 1274. Retaining arms 1278 are flexible in the radial direction and may be flexed radially outward. The flexibility allows tabs 1280 to be temporarily removed from the longitudinal bore 1276 to permit insertion and removal of inner body 1168 during the assembly or disassembly of guide member 1128. While the present embodiment utilizes two tabs 1280 positioned 180° apart, a different number of tabs may be used, provided they are configured to prevent inner body 1168 from slipping out of outer tubular member 1166. Although the stop shoulder 1170 and retaining arms 1278 are described as integral parts of the outer tubular member, it should be understood that those features may be created by separate elements such as threaded caps or spring clips. As a further alternative, where a removable cap or clip is used, the retaining arms may be replaced by a second annular wall.

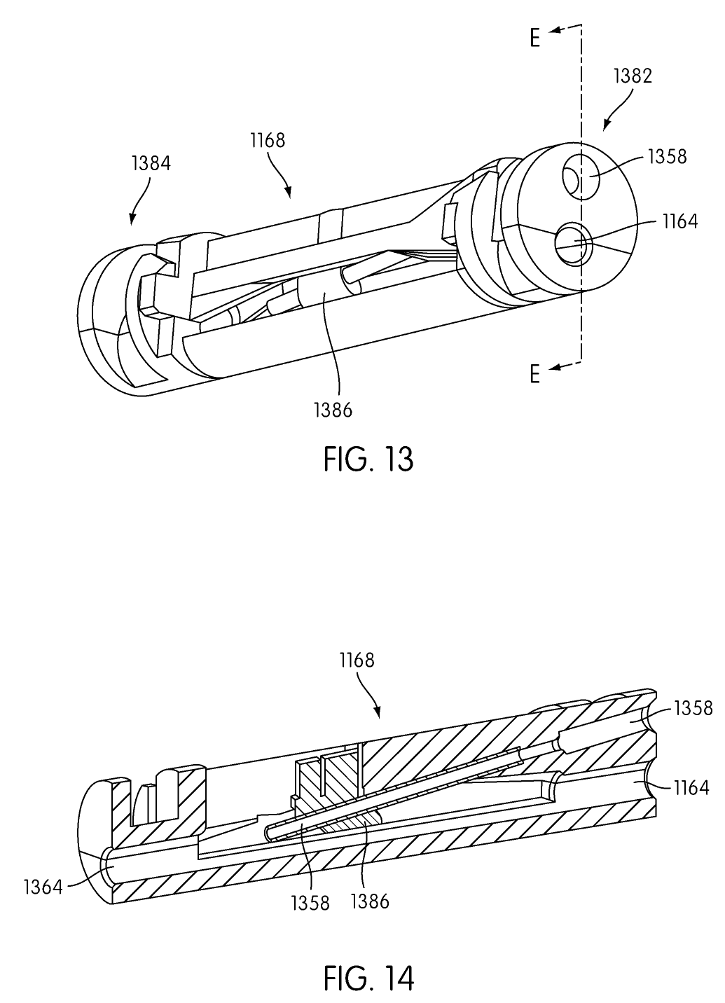

Inner body 1168, as shown in greater detail in FIGS. 13 and 14, generally functions as guide member 828, of the embodiment previously described. Inner body 1168 has proximal and distal ends, 1382 and 1384 respectively. A catheter receiving bore 1164 extends longitudinally through inner body 1168 from proximal end 1382 to distal end 1384. In the present embodiment, unlike the embodiment shown in FIGS. 8-10, guide member 1128 employs a single keel spreader member 1386. Keel spreader member 1386 serves to locally spread open guideway 1150 when guide member 1128 is slideably mounted on elongated shaft 1102. Guidewire passageway 1358 extends through inner body 1168 such that its distal-most end intersects catheter receiving bore 1164 at a shallow angle, preferably ranging from 3° to 15°. Guidewire passageway 1358 extends through keel spreader member 1386 to assure that guidewire 1114, may travel unobstructed through guideway 1150.

It shall be understood that the single keel design may be substituted for the dual spreader design, shown in FIG. 9, and vice versa. In addition, like guide member 828, guide member 1128 may be molded from a rigid plastic material, such as nylon or nylon based co-polymers, that is preferably lubricious. Alternatively, guide member 1128 may be made of a suitable metal, such as stainless steel, or guide member 1128 may have both metal components and plastic components. For ease in manufacturing, guide member 1128 may be comprised of molded parts that snap-fit together to form the final configuration.

A further alternative embodiment to the guide members 128, 428 discussed above is illustrated in FIGS. 15-17. In this embodiment, guide member 1528 provides direct control over axial movement of indwelling guidewire 1614 by providing a clamping mechanism that releasably couples guidwire 1614 and guide member 1528. One such guide member is disclosed in U.S. Patent Application Publication 2004-0039372 A1, published Feb. 26, 2004, the disclosure of which is incorporated by reference in its entirety herein.

As shown in FIG. 16, guide member 1528 has a main body having both proximal and distal ends, 1554 and 1556 respectively. A catheter receiving bore 1664 extends longitudinally through guide member 1528 from proximal end 1554 to distal end 1556. Guide member 1528 includes a proximal spreader member 1660 and a distal spreader member 1662 extending radially into catheter receiving bore 1664. In addition, a tubular guidewire receiver 1694 is mounted to proximal and distal spreader members, 1660 and 1662 respectively, within catheter receiving bore 1664 and is sized to slideably receive guidewire 1614. The pair of spreader members serve to locally spread open guideway 1550 and provide a structure for holding tubular guidewire receiver 1694 within guidewire lumen 1632 when guide member 1528 is slideably mounted on elongated shaft 1502. Tubular guidewire receiver 1694 has a side opening 1690 sized to receive a clamp member 1696. Proximal spreader member 1660 and distal spreader member 1662 serve to align elongated shaft 1502 within catheter receiving bore 1664 and to align guideway 1550 with side opening 1690 on tubular guidewire receiver 1694.

Clamp member 1696 extends radially inward from a clamp control member 1530. Clamp control member 1530 and clamp member 1696 extend through the guide member 1528 and allow a clinician to manually engage a clamping force on guidewire 1614. In the present embodiment, a clamp spring 1692 is mounted to clamp control member 1530 and guide member 1528. Clamp spring 1692 holds clamp member 1696 and clamp control member 1530 in a disengaged state when no external force is placed on clamp control member 1530. When clamp control member 1530 is pressed and clamp spring 1692 is compressed, it causes clamp member 1696 to extend further radially into the catheter receiving bore 1664, through side opening 1690 in tubular guidewire receiver 1694 and against guidewire 1614. That engagement with guidewire 1614 results in a frictional force that resists relative movement between guidewire 1614 and guide member 1528, allowing a clinician to directly control the axial location of guidewire 1614 within elongated shaft 1502.

Like guide members 828 and 1128, guide member 1528 may be molded from a rigid plastic material, such as nylon or nylon based copolymers, that is preferably lubricous. Alternatively, guide member 1528 may be made of a suitable metal, such as stainless steel, or guide member 1528 may have both metal components and plastic components. For ease in manufacturing, guide member 1528 may be comprised of molded parts that snap-fit together to form the final configuration.

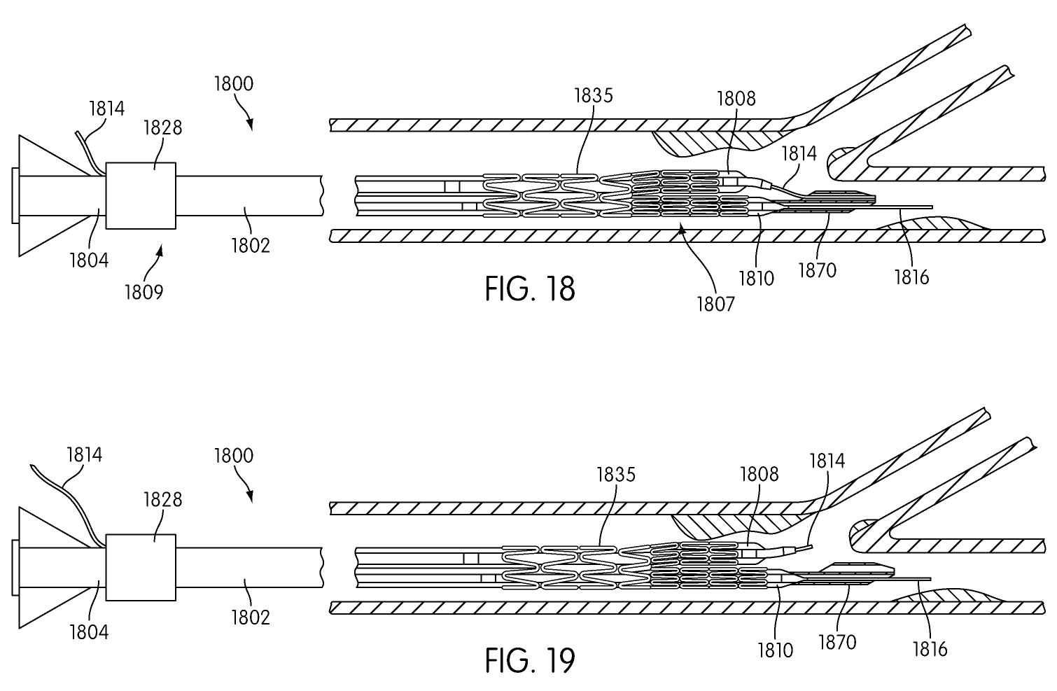

In an embodiment of the present invention, a catheter 1800 configured to deliver a bifurcated stent 1835 to a bifurcated lumen is provided. As illustrated in FIG. 18, the catheter 1800 includes an elongated shaft 1802 that includes a proximal portion 1809 and a distal portion 1807. The distal portion 1807 may have the same or substantially the same configuration as the distal portion of the catheter disclosed in U.S. Pat. No. 6,129,738 to Lashinski et al., which is incorporated herein by reference in its entirety, while the remainder of the catheter 1800 may have a configuration similar to the catheter 100 described above.

The catheter 1800 may be preloaded with a first guidewire 1814 via a guide member 1828, such as the guide member 1528 of the type illustrated in FIGS. 15-17 and described above. The first guidewire 1814 may be tracked through a first branch 1808 of the distal portion 1807 of the catheter 1800 so that a distal tip of the first guidewire 1814 extends just distal of the first branch 1808 and into a distal tip structure 1870 of the type disclosed in U.S. Pat. No. 6,129,738. Once the first guidewire 1814 is in place, the guide member 1528 may be slid to a the proximal end 1804 of the elongated shaft 1802 and locked in place so that the first guidewire 1814 is also locked to the elongated shaft 1802.

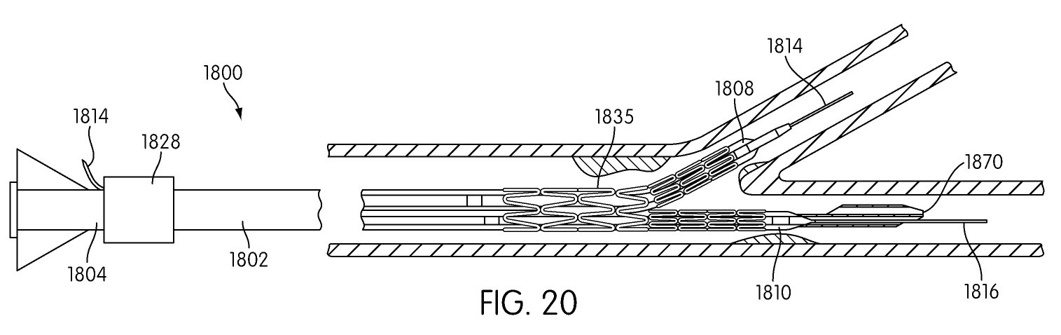

A second guidewire 1816 that has already been tracked to and placed in the main branch of the bifurcated lumen may be front loaded into a second branch 1810 of the catheter 1800 and out an opening (like the opening 122 shown in FIG. 1) of the catheter 1800 so that the catheter 1800 may be tracked to the bifurcation. Once the catheter 1800 is positioned just proximal to the bifurcation in the lumen, the first guidewire 1814 may be tracked to the appropriate position in the side branch, as shown in FIGS. 19 and 20, and the catheter 1800 may be further advanced so that the first branch 1808 of the catheter 1800 is positioned in the side branch of the bifurcated lumen, while the second branch 1810 of the catheter 1800 is positioned in the main branch of the lumen. Once the first branch 1808 and the second branch 1810 are properly positioned, the stent 1835 may be deployed by known methods. To retract the catheter 1800, the guide member 1828 may be unlocked and moved distally along the elongated shaft 1802 and the elongated shaft 1802 may be walked off both guidewires 1814, 1816 simultaneously.

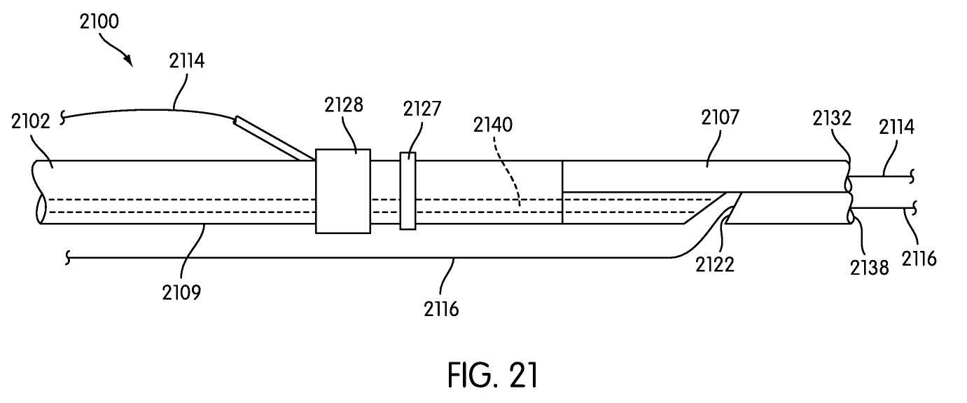

FIG. 21 illustrates an embodiment of a wire placement catheter 2100 that is configured to place two guidewires in a bifurcated lumen, i.e., a first guidewire 2114 into a side branch of the bifurcated lumen and a second guidewire 2116 into a main branch of the bifurcated lumen, without allowing the guidewires 2114, 2116 to wrap around or become entangled with each other. The catheter 2100 includes an elongated shaft 2102 having a proximal portion 2109 and a distal portion 2107. The proximal portion 2109 of the elongated shaft 2102 may include a stiffening member 2140 that is configured to stiffen the proximal portion 2109 of the elongated shaft 2102 to prevent kinking as the catheter is advanced in the lumen to the bifurcation. The distal portion 2107 of the elongated shaft 2102 includes two guidewire lumens, including a first guidewire lumen 2132 for receiving the first guidewire 2114, and a second guidewire lumen 2138 for receiving the second guidewire 2116. An opening 2122 is provided in the distal portion 2107 of the elongated shaft 2122 and is configured to allow the second guidewire 2116 to exit therethrough. A guide member 2128 that is configured to enclose the first guidewire 2114 in the proximal shaft 2102 may be constructed and arranged like any of the guide members described above, such as the guide member 1528 illustrated in FIGS. 15-17.

In operation, the second guidewire 2116 may be tracked to the main branch of the bifurcated lumen. The catheter 2100 may then be front loaded onto the second guidewire 2116 such that the second guidewire 2116 passes through the second lumen 2138 and out of the opening 2122. The guide member 2128 may be slid to a stop member 2127 on the elongated shaft 2102 so that it is positioned to allow the first guidewire 2114 to be loaded into first lumen 2132 of the elongated shaft 2102. Once the catheter 2100 is positioned just proximal to the bifurcation in the lumen, the guide member 2128 may be used to insert the first guidewire 2114 into the first lumen 2132. The first guidewire 2114 may be advanced to the distal end of the catheter 2100, and tracked into the side branch of the bifurcated lumen.

In an embodiment, the first guidewire 2114 may be loaded into the elongated shaft 2102 before the catheter 2100 is tracked to the bifurcation via the second guidewire 2116. In addition, marker bands may be provided to the distal end of the elongated shaft 2102 to assist in visualization of the location and orientation of the distal end of the catheter 2100.

Once both guidewires 2114, 2116 are in place, the catheter 2100 may be removed from the lumen while the guidewires 2114, 2116 are held in place. A stent delivery catheter that includes a stent to be delivered to the bifurcated lumen, may then be front loaded onto each of the guidewires and tracked to the bifurcation, without the guidewires 2114, 2116 becoming entangled.

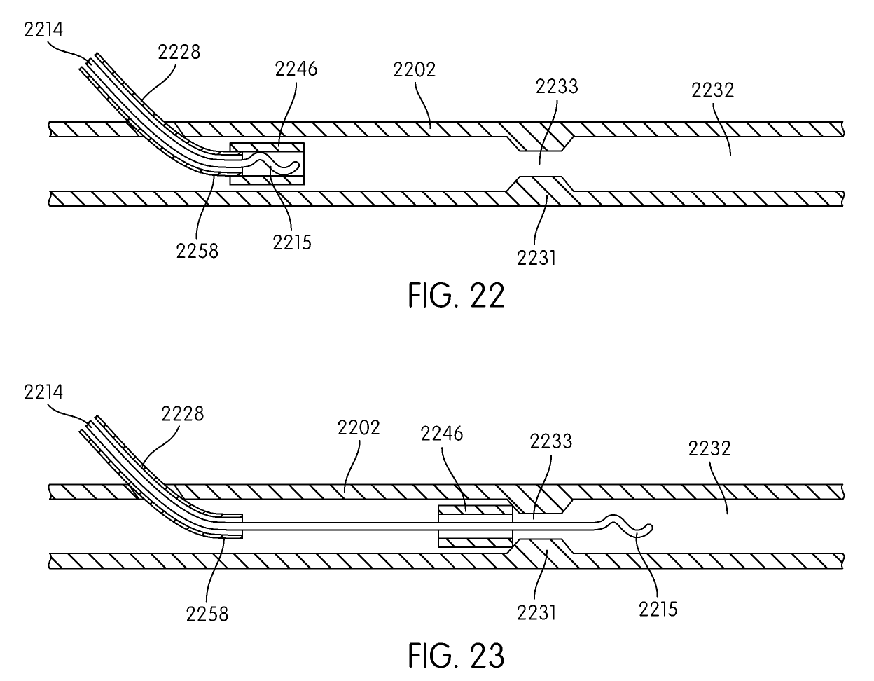

FIGS. 22 and 23 illustrate an embodiment of a shuttle member 2246 that is configured to prevent a guidewire 2214 from puncturing the elongated shaft 2202 while being loaded into a lumen 2232 via a guide member 2228. The shuttle member 2246 may have a generally cylindrical shape and may be slightly flared at a proximal end so that the shuttle member 2246 may form an interference fit with a guidewire passageway 2258 of the guide member 2228, as shown in FIG. 22. A distal end 2215 of the guidewire 2214 may be shaped such that as the distal end 2215 passes through the guidewire passageway 2258 and into the lumen 2232 of the elongated shaft 2202, the distal end 2215 of the guidewire 2214 may be captured by the shuttle member 2246, as shown in FIG. 22. As the guidewire 2214 is pushed towards a distal end of the elongated shaft 2202, the shuttle member 2246 releases from the guidewire passageway 2258 and travels with the distal end 2215 of the guidewire 2214 in the lumen 2232.

As shown in FIGS. 22 and 23, the lumen 2232 includes a narrow section 2233 that may be defined by a radial protrusion 2231. The narrow section 2233 is large enough to allow the shaped distal end 2215 of the guidewire 2214 to pass through, but is too narrow to allow the shuttle member 2246 to pass through. Instead, the shuttle member 2246 abuts the radial protrusion 2231, and as force continues to be applied to the guidewire 2214 to advance the guidewire 2214 through the lumen 2232, the shaped distal end 2215 of the guidewire 2214 exits the shuttle member 2246 and continues through the narrow section 2233 and to the distal end of the elongated shaft 2202. Conversely, when the guidewire 2214 is pulled in the proximal direction, the shaped distal end 2215 of the guidewire 2214 may pass through the narrow section 2233 and back into the shuttle member 2246, which may capture the distal end 2215 and travel with the distal end 2215 of the guidewire 2214 back to the guidewire passageway 2258, where it may attach itself to the guidewire passageway 2258 via an interference fit. The ability for the shuttle member 2246 to travel back and forth between the guidewire passageway 2258 and the narrow section 2233 of the lumen 2232 allows the operator to reshape the distal end 2214 of the guidewire 2214 if desired.

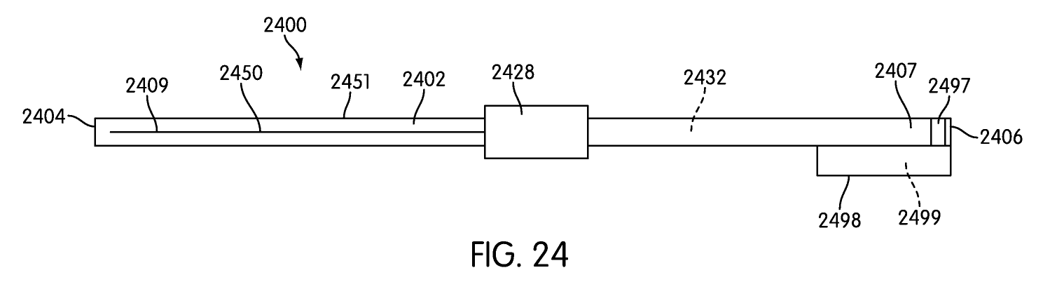

FIGS. 24-26 illustrate an embodiment of a guidewire placement catheter 2400 that is configured to allow two guidewires to be tracked into position in a bifurcated lumen without guidewire entanglement. By being able to place two guidewires into position without entanglement, more conventional over-the-wire type bifurcated catheter delivery systems may be delivered to bifurcated lumens more easily. As illustrated, the guidewire placement catheter 2400 includes an elongated shaft 2402 that has a proximal end 2404 and a distal end 2406. The elongated shaft 2402 has a single lumen 2432 that extends from the proximal end 2404 to the distal end 2406. The lumen 2432 is configured, i.e., sized and shaped, to receive a guidewire 2414. Similar to embodiments of the elongated shafts described above, at least a proximal section 2409 of the elongated shaft 2402 may include a guideway 2450 that connects an outer surface 2451 of the proximal section 2409 of the elongated shaft 2402 to the lumen 2432.

A guide member 2428, such as one of the guide members described above, is configured to slide on the outer surface 2451 of the proximal portion 2409 of the elongated shaft 2402. The guide member 2428 includes a spreader member, such as a keel spreader member described above, that is constructed and arranged to extend into the guideway 2450 and create a gap through which the guidewire 2413 may pass into and out of the lumen 2432, as described above in other embodiments.

The guidewire placement catheter 2400 also includes a tracking section 2498 that is connected to the elongated shaft 2402 at the distal portion 2407 thereof. The tracking section 2498 includes a passageway 2499 that is configured to receive a second guidewire 2416. The passageway 2499 is substantially parallel to the lumen 2432 so that the guidewires 2414, 2416 may be substantially parallel to one another as the guidewire placement catheter is advanced. A marker band 2497 may be placed on or in the distal end 2406 of the elongated shaft 2402 and/or the tracking section 2498 so as to allow visualization of the distal end 2406 of the guidewire placement catheter 2400 as the guidewire placement catheter 2400 is advanced in the bifurcated lumen. Fabrication and use of such marker bands are known and therefore are not described in greater detail herein.

To use the guidewire placement catheter 2400, the guidewire 2416 is first advanced into a main branch of a bifurcated lumen by known methods. Once the guidewire 2416 is in place, a proximal end of the guidewire may be back loaded into the passageway 2499 of the tracking section 2498 of the guidewire placement catheter 2400. The other guidewire 2414 may then be front loaded into the lumen 2432 of the elongated shaft 2402 via the guide member 2428.

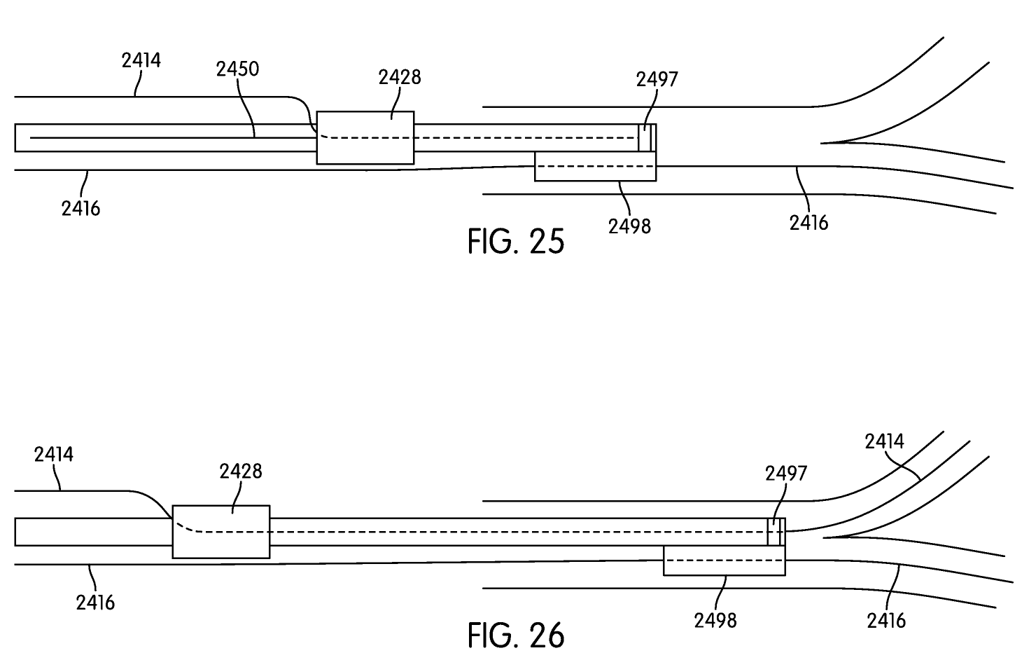

If the guide member 2428 is in the most distal position on the guidewire placement catheter 2400, then the guidewire placement catheter 2400 may then be advanced to the bifurcation in the lumen while maintaining the position of the guidewire 2416, as shown in FIG. 25. As the guidewire placement catheter 2400 is advanced, the guidewire 2414 may continue to be loaded via the guide member 2428 by moving the guide member 2428 towards the proximal end 2404 of the elongated shaft 2402. This may be done incrementally. For example, the guidewire placement catheter 2400 may be advanced a small distance and stopped, then the guidewire 2414 may be advanced a small distance and stopped, and so on. This may continue until the guidewire placement catheter 2400 reaches the bifurcation

If the guide member 2428 is in the most proximal position on the guidewire placement catheter 2400, then the guidewire 2414 may be front loaded up to the distal end 2406 of the catheter 2400. The guidewire placement catheter 2400 with guidewire 2414 in-situ in the catheter lumen may then advanced over guidewire 2416 until it reached the bifurcated lesion. At this point, the guidewire 2416 may be advanced out the distal end 2406 of guide catheter 2400 into the second branch of the lumen.

When the guidewire placement catheter 2400 reaches the bifurcation, the guidewire 2114 may be advanced into the side branch of the bifurcated lumen, as shown in FIG. 26. The guidewire placement catheter 2400 may then be withdrawn from the bifurcated lumen, while leaving the guidewires 2414, 2416 in place. A stent delivery catheter may then be front loaded onto the guidewires 2414, 2416 and tracked to the bifurcation without having the guidewires cross, wrap, or otherwise entangle.

Although the illustrated embodiments of the guide member show an elongated shaft having a circular cross-section it shall be understood that the guide member may be configured to slidably couple to a catheter shaft having any geometry. For example, the catheter receiving bore of any of the embodiments may be kidney-shaped to receive an elongated shaft having a kidney-shaped cross-section.

While various embodiments of the present invention have been described above, it should be understood that they have been presented by way of example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the following claims and their equivalents. All patents and publications discussed herein are incorporated in their entirety by reference thereto.

What is claimed is:

1. A delivery system for a prosthesis, comprising:
an elongated shaft having a proximal end and a distal end;
a first guidewire lumen disposed in the elongated shaft;
a first guidewire disposed in the first guidewire lumen;
a first guideway disposed in the elongated shaft, the first guideway extending into the first guidewire lumen;
a second guidewire lumen disposed in the elongated shaft;
a second guidewire disposed in the second guidewire lumen;
a first guide member slidably coupled to the elongated shaft, the first guide member being configured to guide the first guidewire through the first guideway and into the first guidewire lumen;
a first branch of the elongated shaft extending from the distal end thereof, the first guidewire extending into the first branch;
a second branch of the elongated shaft extending from the distal end thereof, the second guidewire extending into the second branch;
a first balloon disposed on the first branch;
a second balloon disposed on the second branch;
an inflation lumen disposed in the elongated shaft, the inflation lumen being fluidly connected to at least the first balloon.

2. The delivery system according to claim 1, further comprising
a second inflation lumen disposed in the elongated shaft, the second inflation lumen being fluidly connected to the second balloon.

3. The delivery system according to claim 1, wherein the inflation lumen is fluidly connected to the second balloon.

4. The delivery system according to claim 1, wherein the first guide member includes a clamping mechanism that extends into the first guidewire lumen through the first guideway.

5. The delivery system according to claim 1, wherein the first guide member includes a proximal guidewire passageway that extends into the first guidewire lumen through the first guideway.

6. The delivery system according to claim 1, wherein a second entry port for the second guidewire is disposed distal to the elongated shaft proximal end.

7. The delivery system according to claim 1, wherein a stent is mounted on the first branch and the second branch.

8. The delivery system according to claim 1, wherein the elongated shaft has a kidney-shaped cross section.

9. The delivery system according to claim 1, wherein the elongated shaft has a circular cross section.

10. The delivery system according to claim 1, further comprising a second guideway disposed in the elongated shaft, the second guideway extending into the second guidewire lumen.

11. The delivery system according to claim 10, further comprising:
a second guide member configured to guide the second guidewire through the second guideway and into the second guidewire lumen.

12. The delivery system according to claim 11, wherein the first guide member includes a clamping mechanism that extends into the first guidewire lumen through the first guideway and the second guide member includes a guidewire passageway that extends into the second guidewire lumen through the second guideway.

13. The delivery system according to claim 11, wherein the first guide member includes a first clamping mechanism that extends into the first guidewire lumen through the first guideway and the second guide member includes a second clamping mechanism that extends into the second guidewire lumen through the second guideway.

14. The delivery system according to claim 11, wherein the first guide member includes a first guidewire passageway that extends into the first guidewire lumen through the first guideway and the second guide member includes a second guidewire passageway that extends into the second guidewire lumen through the second guideway.

15. The delivery system according to claim 11, wherein the first guide member and second member are integrated together.

16. The delivery system according to claim 1, further comprising a shuttle constructed and arranged to be slidable within the first guidewire lumen and to receive a distal tip of the first guidewire.

17. The delivery system according to claim 16, wherein the first guidewire lumen comprises a narrow portion configured to allow the distal tip of the guidewire to pass threrethrough but not allow the shuttle to pass therethrough.

18. A guidewire placement catheter, comprising:
an elongated shaft having a proximal end and a distal end;
a first lumen extending from the proximal end to the distal end of the proximal shaft, the first lumen being configured to receive a first guidewire;
a guideway extending from an outer surface of a proximal section of the elongated shaft to the first lumen;
a guide member configured to slide on the outer surface of the proximal portion of the elongated shaft, the guide member having a spreader member constructed and arranged to extend into the guideway and create a gap through which the first guidewire may pass into and out of the lumen; and
a second lumen substantially parallel to the first lumen near the distal end of the elongated shaft, the second lumen being configured to receive a second guidewire.

19. The guidewire placement catheter according to claim 18, further comprising a tracking section connected to the elongated shaft at the distal end of the elongated shaft, the tracking section comprising the second lumen.

20. The guidewire placement catheter according to claim 18, wherein the distal end of the elongated shaft comprises a marker band.

21. The guidewire placement catheter according to claim 18, wherein a proximal section of the elongated shaft comprises a third lumen substantially parallel to the first lumen, and a stiffening member positioned in the third lumen, the stiffening member being configured to provide stiffness to the elongated shaft.

22. The guidewire placement catheter according to claim 21, wherein the stiffening member comprises a stiffening wire.

23. The guidewire placement catheter according to claim 18, further comprising a shuttle constructed and arranged to be slidable within the first lumen and to receive a distal tip of the first guidewire.

24. The guidewire placement catheter according to claim 23, wherein the first lumen comprises a narrow portion configured to allow the distal tip of the guidewire to pass threrethrough but not allow the shuttle to pass therethrough.

25. A method for placing two guidewires into a bifurcated lumen, the method comprising:
advancing a first guidewire into a main branch of a bifurcated lumen;
back loading a proximal end of the first guidewire into a tracking section of a guidewire placement catheter;
front loading a second guidewire into a lumen of the guidewire placement catheter;
advancing the guidewire placement catheter while maintaining the first guidewire position to the bifurcate in the bifurcated lumen; and
advancing the second guidwire into a side branch of the bifurcated lumen.

26. The method of claim 25, wherein the front loading of the second guidewire and the advancing of the guidewire placement catheter are done simultaneously.

27. The method of claim 25, wherein the front loading of the second guidewire and the advancing of the guidewire placement catheter are done incrementally.

28. The method of claim 25, wherein the front loading of the second guidewire comprising inserting the second guidewire into a guide member, and sliding the guide member along a elongated shaft of the catheter so that the second guidewire passes through a guideway in the elongated shaft and into the lumen of the guidewire placement catheter.
